(19) United States
(12) Patent Application Publication
(10) Pub. No.: US 2008/0114029 A1
(43) Pub. Date: May 15, 2008

(54) Polymorphs of solifenacin intermediate

(76) Inventors: Tamas Koltai, Netanya (IL); Nurit Perlman, Kfar Saba (IL); Tamar Nidam, Yehud (IL); Dov Diller, Jerusalem (IL)

Correspondence Address: KENYON & KENYON LLP, ONE BROADWAY, NEW YORK, NY, 10004, US

(21) Appl. No.: 11/890,264

(22) Filed: Aug. 3, 2007

Related U.S. Application Data
(60) Provisional application No. 60/835,806, filed on Aug. 3, 2006.
(60) Provisional application No. 60/845,260, filed on Sep. 18, 2006.
(60) Provisional application No. 60/845,261, filed on Sep. 18, 2006.
(60) Provisional application No. 60/859,951, filed on Nov. 20, 2006.
(60) Provisional application No. 60/859,952, filed on Nov. 20, 2006.
(60) Provisional application No. 60/878,913, filed on Jan. 4, 2007.
(60) Provisional application No. 60/898,789, filed on Jan. 31, 2007.
(60) Provisional application No. 60/898,888, filed on Jan. 31, 2007.
(60) Provisional application No. 60/930,391, filed on May 15, 2007.
(60) Provisional application No. 60/949,112, filed on Jul. 11, 2007.

(51) Int. Cl. A61K 31/47; C07D 217/02

(52) U.S. Cl. 514/307; 546/139

(57) ABSTRACT

Polymorphic forms of 1(S)-phenyl-1,2,3,4-tetrahydroisoquinoline have been prepared and characterized. These polymorphic forms are particularly useful for preparing solifenacin salts.

### CROSS-REFERENCE TO RELATED APPLICATIONS

This application claims the benefit of Provisional Application Ser. No. 60/835,806, filed Aug. 3, 2006, Provisional Application Ser. No. 60/845,260, filed Sep. 18, 2006, Provisional Application Ser. No. 60/845,261, filed Sep. 18, 2006, Provisional Application Ser. No. 60/859,951, filed Nov. 20, 2006, Provisional Application Ser. No. 60/859,952, filed Nov. 20, 2006, Provisional Application Ser. No. 60/878,913, filed Jan. 4, 2007, Provisional Application Ser. No. 60/898,789, filed Jan. 31, 2007, Provisional Application Ser. No. 60/898,888, filed Jan. 31, 2007, Provisional Application Ser. No. 60/930,391, filed May 15, 2007, and to Provisional Application Ser. No. 60/949,112, filed Jul. 11, 2007. The contents of these applications are incorporated herein in their entirety by reference.

### FIELD OF INVENTION

The present invention is related to solid state chemistry of 1-phenyl-1,2,3,4-tetrahydroisoquinoline, which is a useful intermediate for making solifenacin succinate.

### BACKGROUND OF INVENTION

1-Pheny-1,2,3,4-tetrahydroisoquinoline (“IQL”) of the following formula

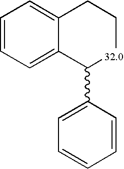

is the key intermediate of making solifenacin salts such as solifenacin succinate. Solifenacin succinate, (3R)-1-azabicyclo[2.2.2]oct-3-yl-(1S)-1-phenyl-3,4-dihydroisoquinoline-2-(1H)-carboxylate-succinate, or 1(S)-phenyl-1,2,3,4-tetrahydroisoquinoline-2-carboxylic acid 3(R)-quinuclidinyl ester succinate of the formula

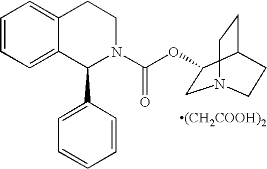

is a urinary antispasmodic indicated for the treatment of urge incontinence and/or increased urinary frequency and urgency as may occur in patients with overactive bladder syndrome (“OAB”). The drug is marketed under the name Vesicare® in 5 mg and 10 mg tablets.

Solifenacin and derivatives thereof, as well as salts thereof, are reported to encompass in U.S. Pat. No. 6,017,927.

U.S. Patent Application Nos. 60/859,952 and 60/949,112 describe the preparation of (S)-IQL tartrate the conversion of 1(S)-phenyl-1,2,3,4-tetraisoquinoline tartrate (“(S)-IQL tartrate”) to 1(S)-phenyl-1,2,3,4-tetraisoquinoline (“(S)-IQL”) by addition of a base.

Polymorphism, the occurrence of different solid state forms, is a property of some molecules and molecular complexes. A single molecule, like solifenacin base, may give rise to a variety of solid state forms having distinct crystal structures and physical properties such as melting point, powder x-ray diffraction (“PXRD”) pattern, infrared (“IR”) absorption fingerprint, and solid state nuclear magnetic resonance (“NMR”) spectrum. One solid state form may give rise to thermal behavior different from that of another solid state form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different solid state forms results from the orientation and intermolecular interactions of adjacent molecules or complexes in the bulk solid. Accordingly, polymorphs are distinct solids sharing the same molecular formula yet having distinct advantageous physical properties compared to other solid state forms of the same compound or complex.

One of the most important physical properties of pharmaceutical compounds is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. Different solid state forms or polymorphs of the same pharmaceutical compounds can and reportedly do have different aqueous solubilities.

The discovery of new polymorphic forms of IQL and salts thereof provides a new opportunity to improve the performance of the synthesis of the active pharmaceutical ingredient (“API”), solifenacin succinate, by producing solid state forms of IQL and salts thereof having improved characteristics, such as flowability. Thus, there is a need in the art for polymorphic forms of IQL and salts thereof.

### SUMMARY OF INVENTION

In one embodiment, the invention encompasses a crystalline form of 1(S)-phenyl-1,2,3,4-tetrahydroisoquinoline (“(S)-IQL”), of the following formula

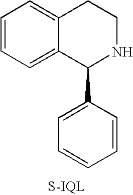

, characterized by PXRD pattern with peaks at about 10.2, 12.4, 15.4, and 16.3±0.2°2θ.

In one embodiment, the invention encompasses a crystalline form of (S)-IQL characterized by PXRD peaks at about 10.2, 12.4, 15.4, and 16.3°±0.2° 2θ containing not more than about 10 wt %, preferably not more than about 5 wt %, and more preferably not more than about 1 wt % of other crystalline form of (S)-IQL.

In another embodiment, the invention encompasses a process for preparing a crystalline form of (S)-IQL characterized by PXRD pattern with peaks at about 10.2, 12.4, 15.4, and 16.3±0.2 °2θ, comprising combining (S)-IQL tartrate with water, an inorganic base, and an organic solvent.

In another embodiment, the invention encompasses a process for preparing a crystalline form of (S)-IQL characterized by PXRD pattern with peaks at about 10.2, 12.4, 15.4, and 16.3±0.2 °2θ, comprising slurrying (S)-IQL tartrate in water and adding an inorganic base.

In yet another embodiment, the invention encompasses a process for preparing a solifenacin salt by preparing according to the processes described above and converting it to a pharmaceutically acceptable salt of solifenacin.

### BRIEF DESCRIPTION OF THE DRAWINGS

FIG. 1 illustrates PXRD pattern of S-IQL characterized by peaks at about 10.2, 12.4, 15.4, and 16.3±0.2°2θ.

### DETAILED DESCRIPTION OF INVENTION

As used herein, the term “room temperature” or “RT” refers to the ambient temperature of a typical laboratory, which is usually about 15° C. to about 30° C., often about 18° C. to about 25° C.

As used herein, the term “vacuum” refers to a pressure of about to 2 mmHg to about 100 mmHg.

As used herein, the term “polymorphic purity” refers to the purity of one polymorphic form with respect to other polymorphic forms, the term “enantiomeric purity” refers to the purity of an enantiomer with respect to the other enantiomer.

As used herein, the term “IQL” refers to 1-pheny-1,2,3,4-tetrahydroisoquinoline, the term “(S)-IQL” refers to 1(S)-phenyl-1,2,3,4-tetraisoquinoline, and the term “(S)-IQL tartrate” refers to 1(S)-phenyl-1,2,3,4-tetraisoquinoline tartrate.

As used herein, the term “PXRD” refers to powder X-ray diffraction.

In one embodiment, the invention encompasses a crystalline form of 1(S)-phenyl-1,2,3,4-tetrahydroisoquinoline, of the following formula

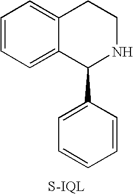

, characterized by PXRD pattern with peaks at about 10.2, 12.4, 15.4, and 16.3±0.2 °2θ.

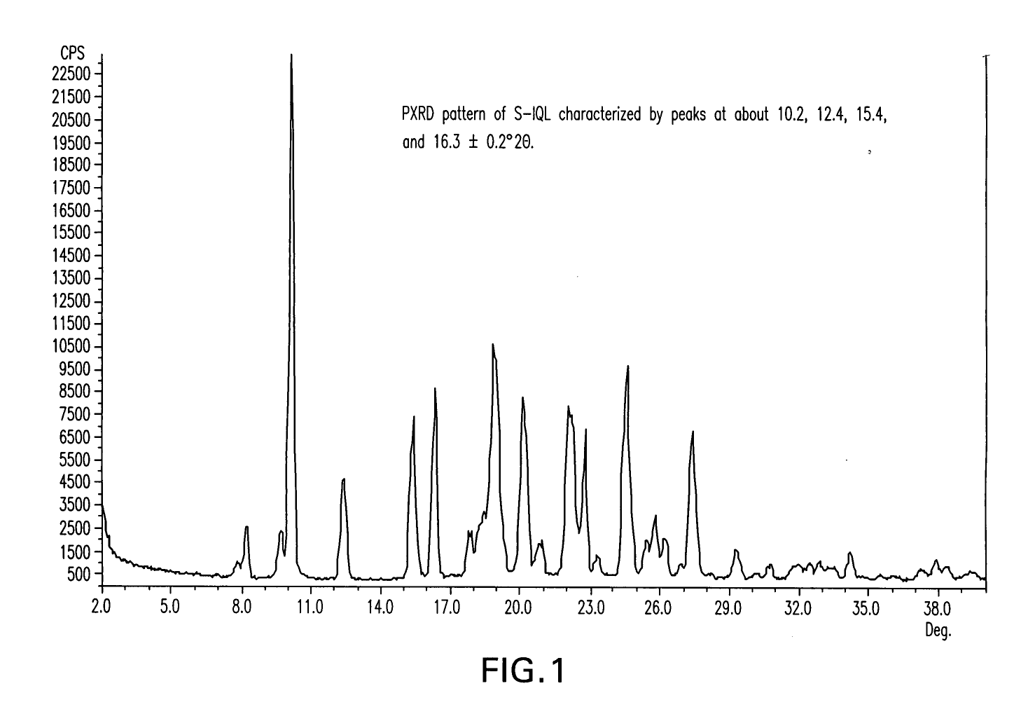

The crystalline form of (S)-IQL may be further characterized by data selected from a group consisting of PXRD pattern with peaks at about 18.9, 20.1, 22.1, 22.8, 24.6, and 17.4±0.2 °2θ and PXRD pattern substantially as depicted in FIG. 1.

Optionally, the above crystalline form of (S)-IQL characterized by PXRD peaks at about 10.2, 12.4, 15.4, and 16.3°±0.2° 2θ containing not more than about 10 wt %, preferably not more than about 5 wt %, and more preferably not more than about 1 wt % of other crystalline form of (S)-IQL. Preferably, the polymorphic purity is measured by PXRD.

In another embodiment, the invention encompasses a process for preparing a crystalline form of (S)-IQL characterized by PXRD pattern with peaks at about 10.2, 12.4, 15.4, and 16.3±0.2 °2θ, comprising combining (S)-IQL tartrate with water, an inorganic base, and an organic solvent.

Optionally, the (S)-IQL tartrate may be obtained according to U.S. Patent Application No. 60/949,112.

Preferably, the inorganic base is selected from the group consisting of NaOH, KOH, NaHCO₃, KHCO₃, Na2CO₃, K₂CO₃, and mixtures thereof. More preferably, the base is NaOH. Preferably, the organic solvent is selected from the group consisting of toluene, THF, and mixtures thereof.

Preferably, the combining step is at about room temperature.

Optionally, after the combination step, a two phase system is obtained. Preferably, the two phases are separated. Preferably, the crystalline form is obtained from the organic phase. Preferably, the crystalline form is obtained by evaporating the organic phase.

In another embodiment, the invention encompasses a process for preparing a crystalline form of (S)-IQL characterized by PXRD pattern with peaks at about 10.2, 12.4, 15.4, and 16.3±0.2 °2θ, comprising slurrying (S)-IQL tartrate in water and adding an inorganic base.

Preferably, the base is selected from the group consisting of NaOH, KOH, NaHCO₃, KHCO₃, Na2CO₃, K₂CO₃, and mixtures thereof. More preferably, the base is NaOH.

Preferably, the base is added gradually to the slurry. Preferably, the base is added to obtain a pH of about 10 to about 14, more preferably of about 12.

Optionally, most of the (S)-IQL is dissolved in water. Optionally, the process further comprises recovering the crystalline form. Preferably, the recovery comprises isolating, washing, and drying. Preferably, the isolation is by filtration. Preferably, the washing is with water. Preferably, the drying is at about 40° C. to about 60° C., more preferably at about 55° C. Preferably, the drying is under vacuum. Preferably, the drying is over night.

In yet another embodiment, the invention encompasses a process for preparing a solifenacin salt by preparing (S)-IQL according to the processes described above and converting it to a pharmaceutically acceptable salt of solifenacin. Preferably, the solifenacin salt is selected from the group consisting of solifenacin succinate, solifenacin acetate, and solifenacin-HX, wherein X is a halogen atom, preferably Cl. More preferably, the solifenacin salt is solifenacin succinate.

(S)-IQL may be converted to (S)-IQL alkyl carbamate by reacting with an alkyl carbamate, for example, according to the methods disclosed in U.S. patent application Ser. No. 11/645,021, which is incorporated herein by reference. (S)-IQL alkyl carbamate may be converted to solifenacin by reacting with 3(R)-quinuclidinol in the presence of base, for example, according to the methods disclosed in U.S. Patent Application No. 60/930,391 and U.S. patent application Ser. No. 11/645,021.

Solifenacin may be converted to solifenacin succinate by reacting with succinic acid, for example, according to the methods disclosed in U.S. Patent Application No. 60/930,391 and U.S. patent application Ser. No. 11/645,021.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way.

### EXAMPLES

X-Ray Powder Diffractions were performed on ARL (Scintag) X-Ray powder diffractometer model X'TRA, Cu-tube, solid state detector. A round standard aluminum sample holder with round zero background quartz plate was used. Scanning parameters: range 2-40° 2θ, continuous scan, rate 5 deg/min.

### Example 1

### Preparation of (S)-IQL From (S)-IQL Tartrate

NaOH (47%, 10 ml) was added into a mixture of (S)-IQL tartrate (23.0 g, enantiomeric purity 99.34%), THF (120 ml), and water (60 ml). After a clear solution was obtained, the phases were separated, and the organic solvent was evaporated to obtain the crystalline form of (S)-IQL described above (13.39 g, 99.9% yield).

### Example 2

### Preparation of Preparation of (S)-IQL from (S)-IQL Tartrate

Wet (S)-IQL tartrate (98 g) was stirred with H₂O (700 ml), NaOH (47%, 31 ml), and toluene (300 ml) for 10 min at RT to obtain two clear phases.

The phases were separated, and the aqueous layer was extracted again with toluene (200 ml). The organic phase was evaporated to obtain the crystalline form of (S)-IQL described above (38.5 g, enantiomeric purity: 98.2%).

### Example 3

### Preparation of (S)-IQL from (S)-IQL Tartrate

Wet (S)-IQL tartrate (7 g) was stirred with H₂O (80 ml) to obtain a light slurry. NaOH (47%) was added gradually to obtain a pH of 12 to obtain massive slurry. The product was isolated by vacuum filtration, washed with H₂O, dried in a vacuum oven at 55° C. over night to obtain the crystalline form of (S)-IQL described above (2.7 g, enantiomeric purity: 98.6%).

1. A crystalline form of 1(S)-phenyl-1,2,3,4-tetrahydroisoquinoline, characterized by a PXRD pattern with peaks at about 10.2, 12.4, 15.4, and 16.3±0.2 °2θ.

2. The crystalline form of 1(S)-phenyl-1,2,3,4-tetrahydroisoquinoline of claim 1, characterized by a PXRD pattern with peaks at about 18.9, 20.1, 22.1, 22.8, 24.6, and 17.4±0.2 °2θ.

3. The crystalline form of 1(S)-phenyl-1,2,3,4-tetrahydroisoquinoline of claim 1, characterized by PXRD pattern substantially as depicted in FIG. 1.

4. The crystalline form of 1(S)-phenyl-1,2,3,4-tetrahydroisoquinoline of claim 1, containing not more than about 10% of other crystalline form of 1(S)-phenyl-1,2,3,4-tetrahydroisoquinoline.

5. The crystalline form of 1(S)-phenyl-1,2,3,4-tetrahydroisoquinoline of claim 4, containing not more than about 5% of other crystalline form of 1(S)-phenyl-1,2,3,4-tetrahydroisoquinoline.

6. The crystalline form of 1(S)-phenyl-1,2,3,4-tetrahydroisoquinoline of claim 5, containing not more than about 1% of other crystalline form of 1(S)-phenyl-1,2,3,4-tetrahydroisoquinoline.

7. A process for preparing a crystalline form of 1(S)-phenyl-1,2,3,4-tetrahydroisoquinoline of claim 1, comprising combining 1(S)-phenyl-1,2,3,4-tetrahydroisoquinoline tartrate with water, an inorganic base, and an organic solvent.

8. The process of claim 7, wherein the inorganic base is selected from the group consisting of NaOH, KOH, NaHCO₃, KHCO₃, Na2CO₃, K₂CO₃, and mixtures thereof.

9. The process of claim 8, wherein the inorganic base is NaOH.

10. The process of claim 7, wherein the organic solvent is selected from the group consisting of toluene, THF, and mixtures thereof.

11. The process of claim 7, wherein the combining is at room temperature.

12. The process of claim 7, wherein after the combination step, a two-phase system of an organic phase and an aqueous phase is obtained.

13. The process of claim 12, wherein the crystalline form is obtained by evaporating the organic phase.

14. A process for preparing a crystalline form of claim 1, comprising slurrying 1(S)-phenyl-1,2,3,4-tetrahydroisoquinoline tartrate in water and adding an inorganic base.

15. The process of claim 14, wherein the base is selected from the group consisting of NaOH, KOH, NaHCO₃, KHCO₃, Na2CO₃, K₂CO₃, and mixtures thereof.

16. The process of claim 14, wherein the base is NaOH.

17. The process of claim 14, wherein the base is added gradually to the slurry.

18. The process of claim 14, wherein the base is added to obtain a pH of about 10 to about 14.

19. The process of claim 18, wherein the base is added to obtain a pH of about 12.

20. The process of claim 14, further comprising recovering crystalline form of 1(S)-phenyl-1,2,3,4-tetrahydroisoquinoline.

21. A process for preparing a solifenacin salt, comprising preparing 1(S)-phenyl-1,2,3,4-tetrahydroisoquinoline according to claim 7 and converting it to a pharmaceutically acceptable salt of solifenacin.

22. The process of claim 21, wherein the solifenacin salt is selected from the group consisting of solifenacin succinate, solifenacin acetate, and solifenacin-HX, wherein X is a halogen atom.

23. The process of claim 22, wherein X is Cl.

24. The process of claim 22, wherein the solifenacin salt is solifenacin succinate.

25. A process for preparing a solifenacin salt, comprising preparing 1(S)-phenyl-1,2,3,4-tetrahydroisoquinoline according to claim 14 and converting it to a pharmaceutically acceptable salt of solifenacin.

26. The process of claim 25, wherein the solifenacin salt is selected from the group consisting of solifenacin succinate, solifenacin acetate, and solifenacin-HX, wherein X is a halogen atom.

27. The process of claim 26, wherein X is Cl.

28. The process of claim 27, wherein the solifenacin salt is solifenacin succinate.
